(19) United States
(12) Patent Application Publication
(10) Pub. No.: US 2021/0345616 A1
(43) Pub. Date: Nov. 11, 2021

(54) METHOD OF PRODUCING METAL NANOPARTICLES AND USES THEREOF

(71) Applicant: RAMOT AT TEL-AVIV UNIVERSITY LTD., Tel Aviv (IL)

(72) Inventors: Shachar RICHTER, Tel Aviv (IL); Roman NUDELMAN, Tel Aviv (IL); Einat HAUZER, Tel Aviv (IL)

(21) Appl. No.: 17/273,857

(22) PCT Filed: Sep. 3, 2019
(86) PCT No.: PCT/IL2019/050984

§ 371 Date: Mar. 5, 2021

Related U.S. Application Data
(60) Provisional application No. 62/727,572, filed on Sep. 6, 2018.

(51) Int. Cl. A01N 59/16; A01N 63/50; A01N 25/12; A01P 1/00; A01P 15/00; A01N 25/34

(52) U.S. Cl. CPC A01N 59/16; A01N 63/50; A01N 25/34; A01P 1/00; A01P 15/00; A01N 25/12

(57) ABSTRACT

The invention disclosed herein relates to a method of producing metal particles of a preselected form in a biological system such a glycoprotein.

### TECHNOLOGICAL FIELD

The present invention generally relates to biological synthesis of metal nanoparticles inside and uses thereof.

### BACKGROUND

Green synthesis of metal nanoparticles in biological agents is an important topic in eco-friendly oriented nanotechnology fields. As it is known, metal nanoparticles such as gold, silver, palladium and others have unique electrical, optical and chemical properties which can be used in various applications in bio-medical and analytical fields of science and industry. There are many well-established chemical and physical procedures for manufacturing metal nanoparticles. Most of these procedures involve the use of organic and inorganic materials, which act as reducers or encapsulation agents. Due to justified ecological reasons, bio-molecules such as proteins can provide a rich environment for research and development of such procedures.

Previous works in the field of green synthesis of metallic nanoparticle teach the use of various bio-molecules for achieving metal nanoparticle formation. Plant, bacteria and fungi extracts were found capable of reducing metal salts with a degree of control over particle formation. Other works describe the use of protein corona as a capping agent or a reducing agent.

Most works that suggest green synthesis of metal particles share several common disadvantages. The first is a lack of understanding of the mechanism which is responsible for the particle formation and second is a lack of control over nanoparticle properties such as size and aggregation. Another problem associated with biological reducing agents is that the identity of the exact molecule which function is to reduce the metal ions often remains unknown.

Mucins glycoproteins are derived from a large family of mucus proteins which are present in various vertebrates and non-vertebrates forms of life. These proteins have a vast array of biological functions, but their most common function is to serve as a protective layer of various organs against external environment. Several types of mucin proteins are well studied because of their related cause to several genetic diseases such as cystic fibrosis. One of the most studied mucin proteins is porcine gastric mucin (PGM) which may be found in a porcine gastric tract. PGM has high molecular weight (2 M0DA-20 MDA) and is approximately 80%-rich in glycosylated oligosaccharide chains. The chains are arranged in 5-15 monosaccharides of galactose, fucose, mannose, N-acetylgalactosamine and N-acetyl glucosamine Those oligosaccharides are attached to the core proteins by O-glycosidic bonds. The rest of the 20% of PGM consist of core proteins which contain a large number of repeated sequences of threonine, serine and proline.

Also, there is a high presence of cysteine-rich regions in hydrophobic pockets along the protein core and in regions which bare similar structures to the von Willebrand factor (vWF) present in red blood cell. The glycoprotein complex of PGM highly resembles tree trunk made of core proteins, to which oligo-saccharides attach in a similar 5fashion to tree branches.

PGM conformational changes under different conditions, mainly pH, were previously studied. Under acidic conditions, pH<4, PGM hydrophobic pockets unfold due5 to breakage of salt bridges present in the inner pocket domain Due to the exposed hydrophobic domain, protein-protein interactions through hydrophobic domains are possible. These interactions form a dense matrix of protein units, which in the natural gastric environment prevent damage to epithelial cells from hydrochloric acid. At pH>4 PGM remains in a folded conformation with hydrophobic pockets closed and protein-protein interactions through electrostatic and hydrogen bonding.

A similar structure and pH dependent conformational modifications are also observed in other mucin glycoproteins such as BSM and recently discovered marine Q-Hend;er, N mucin.

Hendler, N., et al [1] describes a method of synthesizing chiral silver nanoparticles in mucin glycoprotein.

### BACKGROUND ART

[1] Hendler, N., et. al Chemical Communications, 47(26), 7419-7421 (2011)

### SUMMARY OF THE INVENTION

The technology disclosed herein is based on the novel use of various glycoproteins, both commercially obtained (Porcine gastric mucin-PGM, Bovine submaxillary mucin-BSM) as well as naturally obtained (e.g., by extraction from marine organism, namely non-vertebrate organism, such as Q mucin) as reducing and capping agents in green synthesis of various metal particles. By utilizing the glycoprotein pH-dependent conformational states, the inventors developed a method for controlling nanoparticle properties such as size, shape and aggregation for various medical, optical and analytical applications.

Thus, in a most general aspect of the invention, there is provided a method of synthesis of metal particles, e.g., gold, silver, palladium including their alloys or combinations thereof, in a biological matrix comprising at least one glycoprotein. The synthesis utilizes pH-dependent configurational changes imposed to the glycoprotein matrix to thereby control the particles shapes, sizes and aggregation.

In a first aspect, the invention provides a method of producing metal particles, the method comprising affecting a conformational state of at least one glycoprotein enriched with a metal precursor by adjusting/altering the pH of the at least one glycoprotein, thereby causing reduction of the metal precursor to a metal particle of a selected form (size, shape and aggregation).

The invention further provides a method of selective synthesis of metal particles, the method comprising treating a complex of at least one glycoprotein and a metal precursor under a selected pH to thereby selectively produce metal particles of a size, shape and aggregation. The metal particles are provided in a complex with the glycoprotein and may be separated therefrom or used as such.

The invention further provides a method of producing metal particles of a preselected form (size, shape and aggregation), the method comprising reacting a complex of a glycoprotein and a metal precursor with a pH-adjusting agent, e.g., a buffer, under conditions permitting reductive transformation of said metal precursor to metal particles, such that the combination of the glycoprotein, the metal precursor and the pH-adjusting agent determines the form of the metal particle.

The conditions permitting reductive transformation encompass any parameter affecting reduction of the metal precursor to the metal particles. This includes, inter alia, material selection and material concentration, temperature, pH, pressure and volume, excluding the presence of a reducing agent.

In some embodiments, the condition permitting reductive transformation is pH, as disclosed herein. In some embodiments, the pH is acidic and in other embodiments the pH is basic. Generally speaking, the reductive transformation may be achieved at a pH between 3 and 9, or between 3 and 7, or between 3 and 6, or between 7 and 9. In some embodiments, the pH is 3, 4, 5, 6, 8 or 9. In some embodiments, the pH is 3, 6 or 9.

In some embodiments, the pH is selected based on the glycoprotein used. In some embodiments, the pH is selected based on the metal used.

In some embodiments, the transformation is carried out at room temperature or at a temperature between 45 and 70° C.

Alternatively, the selected conditions for carrying out a reductive transformation in accordance with the invention include selecting a combination of pH, metal precursor and glycoprotein. As noted herein, such a selection may determine the size of the metal particles. For example, as exemplified herein, palladium and silver synthesis in PGM under alkaline conditions produces roughly the same particles, i.e., spherical particles having an averaged size of about 10-20 nm. This is due to their similar electronegativity properties and similar atomic numbers. In case of gold particles, under alkaline conditions, spherical particles are also produced but of much greater sizes, e.g., about 50-100 nm.

By adjusting/altering the pH of the glycoprotein environment, conformational changes to the glycoprotein may be induced. These conformational changes determine the size, shape and aggregation capabilities of the produced metal particles. Under acidic conditions, the glycoprotein hydrophobic pockets unfold due to breakage of salt bridges present in the inner pocket domain, exposing the hydrophobic domains and enabling protein-protein interactions. The interactions form a dense matrix of protein units, which permits reductive transformation of the metal precursor into metal particles at the micro-scale, as well as of metal particles on the nanometric scale. At basic pHs, the glycoprotein remains in a folded conformation with the hydrophobic pockets closed, thereby producing metal particles of nanometric sizes.

The conformational state of the glycoprotein may vary from one protein to another, and may additionally change in response to the pH-adjusting conditions, mainly to the nature of the pH-adjusting agent, e.g., buffer, used.

As known in the art, a glycoprotein is to a protein covalently attached to oligosaccharide chains. The glycoprotein used in accordance with the invention may be commercially available or extracted from natural sources, as known in the field. The glycoprotein encompasses any of the family members, including mucins or proteins that are composed from two main components: protein core and oligosaccharide side chains. The oligosaccharide side chains may have different sizes, molecular weights and compositions.

In some embodiments, the glycoprotein is at least one mucin. The mucin may be any member of the mucin family known in the art, such as porcine gastric mucin (PGM), bovine submaxillary mucin (BSM), Q-mucin or any other mucin glycoprotein that is produced and secreted by epithelial cells.

The glycoprotein acts as a matrix for holding the metal precursors and the metal particles produced according to the methods herein. The metal precursor added to the glycoprotein need not be identical to any metal salt form or any metal precursor naturally present in the glycoprotein matrix. The metal precursor may be any charged metal form that is capable of undergoing reduction to the neutral metal particle (metal of zero charge). The “metal precursor” is thus a chemical material which comprises the metal atom in a charged form and which gives rise to the metallic form of the same metal when treated, as disclosed herein, within the glycoprotein matrix. In accordance with the present invention, the metal precursor may be in a form of a metal salt or a metal complex, namely in a non-zero oxidation state, and is transformable to the metallic zero-oxidation state in the glycoprotein matrix, as disclosed herein.

Generally, the metal precursor, e.g., salt or complex, is of a metallic element of Groups VIIB, VIIIB, IB and IIB of block d of the Periodic Table of the Elements. In other embodiments, the metal is a transition metal of Groups VIIB, VIIIB, IB and IIB of block d the Periodic Table. In some embodiments, the transition metal is a metallic element selected from Sc, Ti, V, Mn, Fe, Ni, Cu, Zn, Y, Zr, Nb, Tc, Ru, Rh, Au, Pt, Pd, Ag, Mn, Co, Cd, Hf, Ta, Re, In, Ga, Os and Ir.

In some embodiments, the metal is selected from Cu, Ni, Ag, Au, Pt, Pd, Al, Fe, Co, Ti, Zn, In, Sn and Ga. In some embodiments, the metal is selected from Au, Ag, Pd, Cu, Mn and any alloy thereof.

In some embodiments, the metal precursor is a metal salt. In some embodiments, the metal precursor is a metal complex.

In some embodiments, the metal precursor is a metal salt of Au, Ag, or Pd. In some embodiments, the metal precursor is a metal complex of Au, Ag, or Pd.

In some embodiments, the metal precursor is selected from:

Metal precursors as cations, wherein “M” represents a metal atom as disclosed herein, including:
chlorides, e.g., selected from MCl, MCl₂, MCl₃, MCl₄, MCl₅, and MCl₆;
chlorides hydrates, e.g., selected from MCl.xH₂O, MCl₂.xH₂O, MCl₃.xH₂O, MCl₄.xH₂O, MCl₅.xH₂O, and MCl₆.xH₂O, wherein x varies based on the nature of M;
hypochlorites/chlorites/chlorates/cerchlorates (abbreviated ClOₙ⁻, n=1, 2, 3, 4), e.g., selected from MClOₙ, M(ClOₙ)₂, M(ClOₙ)₃, M(ClOₙ)M(ClOₙ)₅, and M(ClOₙ)₆;
hypochlorites/chlorites/chlorates/perchlorates hydrates, e.g., selected from MClOₙ.xH₂O, M(ClOₙ)₂.xH₂O, M(ClOₙ)₃.xH₂O, M(ClOₙ)₄.xH₂O, M(ClOₙ)₅.xH₂O, and M(ClOₙ)₆.xH₂O, wherein x varies based on the nature of M, and n=1, 2, 3, 4;
carbonates, e.g., selected from M₂CO₃, MCO₃, M₂(CO₃)₃, M(CO₃)₂, M₂(CO₃)₂, M(CO₃)₃, M₃(CO₃)M(CO₃)₅, M₂(CO₃)₇;
carbonate hydrates, e.g., selected from M₂CO₃.xH₂O, MCO₃.xH₂O, M₂(CO₃)₃.xH₂O, M(CO₃)₂.xH₂O, M₂(CO₃)₂.xH₂O, M(CO₃)₃.xH₂O, M₃(CO₃)₄.xH₂O, M(CO₃)₅.xH₂O, and M₂(CO₃)₇.xH₂O, wherein x varies based on the nature of M;
carboxylates (abbreviated RCO₂⁻, and including acetates), e.g., selected from MRCO₂, M(RCO₂)₂, M(RCO₂)₃, M(RCO₂)₄, M(RCO₂)₅, and M(RCO₂)₆;
carboxylates hydrates (abbreviated RCO₂⁻), e.g., selected from MRCO₂.xH₂O, M(RCO₂)₂.xH₂O, M(RCO₂)₃.xH₂O, M(RCO₂)₄.xH₂O, WRCO₂)₅.xH₂O, and M(RCO₂)₆.xH₂O, wherein x varies based on the nature of M;
carboxylate (the group RCOO⁻, R is aliphatic chain, which may be saturated or unsaturated), e.g., selected from CH₃CH═CHCOOM (metal crotonate), CH₃(CH₂)₃CH═CH(CH₂)₇COOM (metal myristoleate), CH₃(CH₂)₅CH═CH(CH₂)₇COOM (metal palmitoleate), CH₃(CH₂)₈CH═CH(CH₂)₄COOM (metal sapienate), CH₃(CH₂)₇CH═CH(CH₂)₇COOM (metal oleate), CH₃(CH₂)₇CH═CH(CH₂)₇COOM (metal elaidate), CH₃(CH₂)₅CH═CH(CH₂)₉COOM (metal vaccinate), CH₃(CH₂)₇CH═CH(CH₂)₁₁COOM (metal erucate), C₁₇H₃₅COOM (metal stearate);
oxides, e.g., selected from M₂O, MO, M₂O₃, MO₂, M₂O₂, MO₃, M₃O₄, MO₅, and M₂O₇;
acetates, e.g., (the group CH₃COO⁻, abbreviated AcO⁻) selected from AcOM, AcO₂M, AcO₃M, and AcO₄M;
acetates hydrates, (the group CH₃COO⁻, abbreviated AcO⁻), e.g., selected from AcOM.xH₂O, AcO₂M.xH₂O, AcO₃M>H₂O, and AcO₄M.xH₂O, wherein x varies based on the nature of M;
acetylacetonates (the group C₂H₇CO₂⁻, abbreviated AcAc⁻), e.g., selected from AcAcM, AcAc₂M, AcAc₃M, and AcAc₄M;
acetylacetonate hydrates (the group C₂H₇CO₂⁻, abbreviated AcAc⁻), e.g., selected from AcAcM.xH₂O, AcAc₂M.xH₂O, AcAc₃M.xH₂O, and AcAc₄M.xH₂O, wherein x varies based on the nature of M;
nitrates, e.g., selected from MNO₃, M(NO₃)₂, M(NO₃)₃, M(NO₃) M(NO₃)₅, and M(NO₃)₆;
nitrates hydrates, e.g., selected from MNO₃.xH₂O, M(NO₃)₂.xH₂O, M(NO₃)₃.xH₂O, M(NO₃)₄.xH₂O, M(NO₃)₅.xH₂O, and M(NO₃)₆.xH₂O, wherein x varies based on the nature of M;
nitrites, e.g., selected from MNO₂, M(NO₂)₂, M(NO₂)₃, M(NO₂) M(NO₂)₅, and M(NO₂)₆;
nitrites hydrates, e.g., selected from MNO₂.xH₂O, M(NO₂)₂.xH₂O, M(NO₂)₃.xH₂O, M(NO₂)₄.xH₂O, M(NO₂)₅.xH₂O, and M(NO₂)₆.xH₂O, wherein x varies based on the nature of M;
cyanates, e.g., selected from MCN, M(CN)₂, M(CN)₃, M(CN)₄, M(CN)₅, M(CN)₆;
cyanates hydrates, e.g., selected from MCN.xH₂O, M(CN)₂.xH₂O, M(CN)₃.xH₂O, M(CN)₄.xH₂O, M(CN)₅.xH₂O, and M(CN)₆.xH₂O, wherein x varies based on the nature of M;
sulfides, e.g., selected from M₂S, MS, M₂S₃, MS₂, M₂S₂, MS₃, M₃S₄, MS₅, and M₂S₇;
sulfides hydrates, e.g., selected from M₂S.xH₂O, MS.xH₂O, M₂S₃.xH₂O, MS₂.xH₂O, M₂S₂.xH₂O, MS₃.xH₂O, M₃S₄.xH₂O, MS₅.xH₂O, and M₂S₇.xH₂O, wherein x varies based on the nature of M;
sulfites, e.g., selected from M₂SO₃, MSO₃, M₂(SO₃)₃, M(SO₃)₂, M₂(SO₃)₂, M(SO₃)₃, M₃(SO₃)₄, M(SO₃)₅, and M₂(SO₃)₇;
sulfites hydrates selected from M₂SO₃.xH₂O, MSO₃.xH₂O, M₂(SO₃)₃.xH₂O, M(SO₃)₂.xH₂O, M₂(SO₃)₂.xH₂O, M(SO₃)₃.xH₂O, M₃(SO₃)₄.xH₂O, M(SO₃)₅.xH₂O, and M₂(SO₃)₇.xH₂O, wherein x varies based on the nature of M;
hyposulfite, e.g., selected from M₂SO₂, MSO₂, M₂(SO₂)₃, M(SO₂)₂, M₂(SO₂)₂, M(SO₂)₃, M₃(SO₂)₄, M(SO₂)₅, and M₂(SO₂)₇;
hyposulfite hydrates, e.g., selected from M₂SO₂.xH₂O, MSO₂.xH₂O, M₂(SO₂)₃.xH₂O, M(SO₂)₂.xH₂O, M₂(SO₂)₂.xH₂O, M(SO₂)₃.xH₂O, M₃(SO₂)₄.xH₂O, M(SO₂)₅.xH₂O, and M₂(SO₂)₇.xH₂O, wherein x varies based on the nature of M;
sulfate, e.g., selected from M₂SO₃, MSO₃, M₂(SO₃)₃, M(SO₃)₂, M₂(SO₃)₂, M(SO₃)₃, M₃(SO₃)₄, M(SO₃)₅, and M₂(SO₃)₇;
sulfate hydrates, e.g., selected from M₂SO₃.xH₂O, MSO₃.xH₂O, M₂(SO₃)₃.xH₂O, M(SO₃)₂.xH₂O, M₂(SO₃)₂.xH₂O, M(SO₃)₃.xH₂O, M₃(SO₃)₄.xH₂O, M(SO₃)₅.xH₂O, and M₂(SO₃)₇.xH₂O, wherein x varies based on the nature of M;
thiosulfate, e.g., selected from M₂S₂O₃, MS₂O₃, M₂(S₂O₃)₃, M(S₂O₃)₂, M₂(S₂O₃)₂, M(S₂O₃)₃, M₃(S₂O₃)₄, M(S₂O₃)₅, and M₂(S₂O₃)₇;
thioulfate hydrates, e.g., selected from M₂S₂O₃.xH₂O, MS₂O₃.xH₂O, M₂(S₂O₃)₃ .xH₂O, M(S₂O₃)₂.xH₂O, M₂(S₂O₃)₂.xH₂O, M(S₂O₃)₃ .xH₂O, M₃(S₂O₃)₄.xH₂O, M(S₂O₃)_{5.}.xH₂O, and M₂(S₂O₃)₇.xH₂O, wherein x varies based on the nature of M;
dithionites, e.g., selected from M₂S₂O₄, MS₂O₄, l M₂(S₂O₄)₃, M(S₂O₄)₂, M₂(S₂O₄)₂, M(S₂O₄)₃, M₃(S₂O₄)₄, M(S₂O₄)₅, and M₂(S₂O₄)₇
dithionites hydrates, e.g., selected from M₂S₂O₄. xH₂O, MS₂O₄.xH₂O, M₂(S₂O₄)₃.xH₂O, M(S₂O₄)₂.xH₂O, M₂(S₂O₄)₂.xH₂O, M(S₂O₄)₃ .xH₂O, M₃(S₂O₄)₄.xH₂O, M(S₂O₄)₅.xH₂O, and M₂(S₂O₄)₇.xH₂₃O, wherein x varies based on the nature of M;
phosphates, e.g., selected from M₃PO₄, M₃(PO₄)₂, MPO₄, and M₄(PO₄)₃;
phosphates hydrates, e.g., selected from M₃PO₄.xH₂O, M₃(PO₄)₂.xH₂O, M5PO₄.xH₂O, and M₄(PO₄)₃.xH₂O, wherein x varies based on the nature of M;

Metal alkyls;

Metal alkoxides;

Metal amines;

Metal phosphines;

Metal thiolates;

Combined cation-anion single source precursors, i.e., molecules that include both cation and anion atoms, for example of the formula M(E₂CNR₂)₂ (M=is a metal, E=is for example a chalcogenide, and R=alkyl, amine alkyl, silyl alkyl, phosphoryl alkyl, phosphyl alkyl).

In some embodiments, the metal precursor is a metal salt selected from AgNO₃, PdCl₂, PtBr₂, PtCl₂, PtCl₄, H₂Pt(OH)₆, Pt(NH₃)₂Cl₄HAuClH₄, NaAuClH₄, AuCl, AuCl₂, AuCl₃, Ag(NH₃)₂]Cl, [Ag(S₂O₃)]Cl, [Ag(CN₂)]Cl, AgF₂, Au(OH)₃, KAuCl₄ and AuBr₃.

In some embodiments, the metal precursor is a slat of an organic acid, e.g., a carboxylate as defined herein, e.g., CH₃COOM, wherein M is for example Ag.

The metal precursor/glycoprotein complex may be formed, as exemplified herein, by first mixing an amount of the metal precursor in a medium containing the glycoprotein. Once the complex is formed, it may be treated with a pH-adjusting agent. The pH-adjusting agent may be an acid or a base, a solution containing an acid or a base or a buffer solution of a specific pH. As known, a “buffer” or a buffer solution contains a mixture of a weak acid and its corresponding base or a weak base and its corresponding acid and enables affecting pH changes to solutions to which it is added. The buffer solution may be used as the medium into which the metal precursor/glycoprotein complex is added and can be selected based on the desired pH and the chemical components making up the buffer solution. In some embodiments, the buffer is a borate buffer, a glycine buffer, a sodium acetate buffer, a citrate buffer, a phosphate-citrate buffer or a glycine-sodium hydroxide buffer.

For achieving optimal or predetermined/preselected particle populations, as may be the case, the buffer solution may be selected based on the glycoprotein and metal used. For example, a borate buffer may be used in combination with a glycoprotein such as PGM and Q-mucin to yield silver particles (from any silver precursor). The borate buffer may similarly be used with BSM to yield silver particles (from any silver precursor). Glycine buffer may be used with a glycoprotein such as PGM and Q-mucin to yield gold particles (from any gold precursor).

In some emboldens, excluded from methods and products of the invention are method utilizing BSM, and silver metal precursors when treated under acidic or basic conditions. In some embodiments, the method excluded from the scope of the invention comprises use of BSM and a borate buffer for the production of silver nanoparticles, e.g., of a size between 5 and 20 nm.

The metal particles produced by a green method of the invention comprise the metal atom of the metal precursor in a zero charge state. The metal particles formed are held within hydrophobic pockets in the glycoprotein, and while may be separated therefrom, the glycoprotein/metal particles are typically used as produced. Depending on the method conditions described herein, the particles may be nanoparticles, namely having an averaged dimeter (if spherical or substantially spherical) or averaged size (measured at the longest axis) at the nanoscale, or microparticles, having an averaged dimeter (if spherical or substantially spherical) or averaged size (measured at the longest axis) in the micrometer scale.

As disclosed, the size of the metal particles may be preselected or adjusted by 4selecting one or more of pH (e.g., by selecting a specific buffer), the glycoprotein, and metal precursor (e.g., by selecting the metal and/or the specific counter ion or ligands, in case of a metal salt or a metal complex, respectively). The selected particles may be in the nanoscale, having an effective diameter of up to 1,000 nm. In some embodiments, the nanometric size is between 5 and 1,000 nm, 5 and 900 nm, 5 and 800 nm, 5 and 700 nm, 5 and 600 nm, 5 and 500 nm, 5 and 450 nm, 5 and 400 nm, 5 and 350 nm, 5 and 300 nm, 5 and 250 nm, 5 and 200 nm, 5 and 150 nm, 5 and 100 nm, 5 and 90 nm, 5 and 85, nm, 5 and 80 nm, 5 and 75 nm, 5 and 70 nm, 5 and 65 nm, 5 and 60 nm, 5 and 55 nm, 5 and 50 nm, 5 and 45 nm, 5 and 40 nm, 5 and 35 nm, 5 and 30 nm, 5 and 25 nm, 5 and 20 nm or 5 and 10 nm. In some embodiments, the nanometric size is between about 20 and 500 nm, 20 and 450 nm, 20 and 400 nm, 20 and 350 nm, 20 and 300 nm, 20 and 250 nm, 20 and 200 nm, 20 and 150 nm, 20 and 100 nm, 20 and 90 nm, 20 and 80 nm, 20 and 70 nm, 20 and 60 nm, 20 and 50 nm or 20 and 40 nm. In some embodiments, the nanometric size is between 50 and 500 nm, 50 and 450 nm, 50 and 400 nm, 50 and 350 nm, 50 and 300 nm, 50 and 250 nm, 50 and 200 nm, 50 and 150 nm or 50 and 100 nm. In some embodiments, the nanometric size is between 200 and 900 nm, 200 and 800 nm, 200 and 700 nm, 200 and 600 nm, 200 and 500 nm, 200 and 400 nm or 200 and 300 nm.

In some embodiments, the nanoparticles have a size of between 20 and 50 nm.

In some embodiments, the particles are in the microscale, namely having an effective diameter or size, as defined, greater than 1,000 nm. In some embodiments, the micrometer size is between 1 micron and 5 microns. In some embodiments, the size is between 1 and 5 microns, 1 and 4.5 microns, 1 and 4 microns, 1 and 3.5 microns, 1 and 3 microns, 1 and 2.5 microns, 1 and 2 microns, 1 and 1.5 microns, 1.5 and 5 microns, 2 and 5 microns, 2.5 and 5 microns, 3 and 5 microns, 3.5 and 5 microns, 4 and 5 microns, 1 and 1.1 microns, 1 and 1.2 microns, 1 and 1.3 microns or between 1 and 1.4 microns.

In some embodiments, the microparticles have a size of between 1 and 1.5 microns.

In some embodiments, the method is tailored for obtaining a particle population that consists nanoparticles or microparticles. In some embodiments, the method is tailored for obtaining a particle population that comprises both nanoparticles and microparticles. In such embodiments, where a mixed population is desired and obtained, it may comprise a particle population having particles of sizes ranging from 5 to 1,500 nm (1.5 micron). In some embodiments, the population comprises particles of a size ranging from 500 and 1,500 nm.

In some embodiments, the particle population comprises a nanoparticle population, as defined and selected herein and a microparticle population, as defined and selected herein. In some embodiments, the population comprises nanoparticles having an averaged size between about 20 and 500 nm, 20 and 450 nm, 20 and 400 nm, 20 and 350 nm, 20 and 300 nm, 20 and 250 nm, 20 and 200 nm, 20 and 150 nm, 20 and 100 nm, 20 and 90 nm, 20 and 80 nm, 20 and 70 nm, 20 and 60 nm, 20 and 50 nm or 20 and 40 nm, and a microparticles of a micrometer size between 1 and 2 microns, 1 and 1.5 microns, 1 and 1.4 microns, 1 and 1.3 microns, 1 and 1.2 microns or between 1 and 1.1 microns.

Not only the size of the particles may be preselected or tailored by modifying the method conditions, as above. The metal particles may be of a variety of shapes and in aggregated or non-aggregated form. In some embodiments, the particles have random shapes, or are substantially spherical. In other embodiments, the particles may be amorphous or crystalline or may have a distinct trigonal or hexagonal shape. As may be the case, the particle population may comprise a variety of particle sub-populations, differing form one another in size, shape, composition and the presence of aggregates.

Also, by using combinations of metals with different electro-negativities, in different molar concentrations, core-shell nanoparticles may be formed. For example, in the case of a palladium-silver core-shell nanoparticle, the palladium atom has an electronegativity value of 2.2 while silver have an electronegativity value of 1.93. Thus, palladium has a higher affinity to electrons then silver. This causes to palladium to be reduced by the mucin glycoprotein faster than silver. Core/shell systems of other metals may also be formed.

Another example, for achieving particles of non-spherical or non-circular shapes, the pH of the reaction may be reduced to below 4.

The particles may be separated from the glycoprotein by a variety of methods. However, for some applications, the glycoprotein matrix containing the metal particles may be highly preferable. Thus, the invention further contemplates metal particle populations, as defined herein, when produced by a method of the invention as well as glycoprotein matrix embedding, containing, comprising or consisting metal particles. The metal particles may be formed according to a method of the invention and separated from the glycoprotein by treating the glycoprotein/metal particles complex with protein digesting enzymes such as pepsin, piranha solution (containing hydro fluoric acid). After protein matrix digestion nanoparticles may be concentrated by centrifugation and further solvent evaporation. Thus, the invention further provides a method of obtaining metal particle population, the method comprising obtaining a glycoprotein/metal particle complex according to the invention and treating said complex to obtain the free particle population.

The particle population so obtained may be used for any application in which nanoparticles are involved.

The invention further provides a glycoprotein/metal nanoparticle complex or a composition of matter comprising the complex. This composition of matter may be in an amorphous form or may be made into a film or a solid form by methods known in the art. In some embodiments, the composition of matter is associated with at least one polymer to afford a solid substrate or solid film.

In some embodiments, the composition of matter of the invention is any one of those listed in Table 2 below.

In some embodiments, the composition of matter used in a method according to the invention involves a pH-adjusting agent that is a borate buffer, a glycoprotein that is PGM or Q-mucin and a metal precursor is a silver metal precursor. In some embodiments, the composition of matter used in a method according to the invention comprises a pH-adjusting agent that is a glycine buffer, a glycoprotein that is PGM or Q-mucin and a metal precursor that is a gold metal precursor. In some embodiments, the pH-adjusting agent is a borate buffer, the glycoprotein is PGM or Q-mucin and the metal precursor is a palladium metal precursor. In some embodiments, the glycoprotein is M-Qmucin, the metal precursor is a silver metal precursor and the pH-adjusting agent is a borate buffer. In some embodiments, the glycoprotein is PGM, the metal precursor is a silver metal precursor and the pH-adjusting agent is a borate buffer. In some embodiments, the glycoprotein is M-Qmucin, the metal precursor is a gold metal precursor and the pH-adjusting agent is a glycine buffer. In some embodiments, the glycoprotein is PGM, the metal precursor is a gold metal precursor and the pH-adjusting agent is a glycine buffer. In some embodiments, the glycoprotein is PGM, the metal precursor is a palladium metal precursor and the pH-adjusting agent is a borate buffer. In some embodiments, the glycoprotein is M-Qmucin or PGM, the metal precursor is a combination of two or more metal precursors and the pH-adjusting agent is a borate buffer or a glycine buffer.

In some embodiments, the composition of matter is:
Q-mucin and a population of gold particles, 0.5 μm-1.5 μm and/or 10 nm-100 nm in size; or
PGM and a population of gold particles, 0.5 μm-1.5 μm and/or 10 nm-100 nm in size.

The composition of matter, as such, or as a film or in the form of a gel or a paste, may be used in a wide variety of applications, depending, inter alia, on the metal used. Such applications include:
Where the composition of matter comprises silver nanopartilces, it can be used as antibacterial materials in wound dressings, surface coatings, textile industry, etc.
Where the composition of matter comprises gold nanoparticles, coupled with laser irradiation it can be used as selective antibacterial and anti biofilm material.
Where the composition of matter comprises gold nanoparticles in solution or embedded in a nanofibrous matrix, coupled with laser irradiation or solar irradiation it can be used as water purification and a desalination material as standalone material or part of a water purification or a desalination device.

Additionally, the composition of matter can be used as catalysts for carbon monoxide oxidation, may be incorporated in sanitaizing materials, in green energy applications and other uses.

Items of the invention are provided as follows:
1. A method of producing metal particles of a preselected form, the method comprising reacting a combination of a glycoprotein and a metal precursor with a pH-adjusting agent, under conditions permitting reductive transformation of said metal precursor to metal particles, such that the combination of the glycoprotein, the metal precursor and the pH-adjusting agent determines the form of the metal particle.
2. The method according to item 1, wherein the metal particle form is selected from particle size, shape and aggregation.
3. The method according to any preceding item, for producing metal particles of a preselected particle size, the method comprising reacting a complex of a glycoprotein matrix and a metal precursor with a pH-adjusting agent, under conditions permitting reductive transformation of said metal precursor to metal particles of a preselected size.
4. The method according to any preceding item, wherein the conditions permitting reductive transformation comprise selecting at least one glycoprotein, at least one pH-adjusting agent, a temperature and pH.
5. The method according to any preceding item, wherein the pH is between 3 and 9, or between 3 and 7, or between 3 and 6, or between 7 and 9.
6. The method according to any preceding item, wherein the pH is 3, 4, 5, 6, 8 or 9.
7. The method according to any preceding item, wherein the pH is selected based on the glycoprotein or based on the metal precursor used.
8. The method according to any preceding item, wherein the conditions comprise reductive transformation at room temperature or at a temperature between 45 and 70° C.
9. The method according to any preceding item, wherein the glycoprotein is at least one mucin.
10. The method according to any preceding item, wherein the mucin is porcine gastric mucin (PGM), bovine submaxillary mucin (BSM), or Q-mucin.
11. The method according to any preceding item, wherein the complex of the glycoprotein and the metal precursor is formed by adding the at least one metal precursor to the glycoprotein.
12. The method according to any preceding item, wherein the metal precursor is a metal salt or a metal complex.
13. The method according to any preceding item, wherein the metal precursor comprises a metal atom selected from Sc, Ti, V, Mn, Fe, Ni, Cu, Zn, Y, Zr, Nb, Tc, Ru, Rh, Au, Pt, Pd, Ag, Mn, Co, Cd, Hf, Ta, Re, In, Ga, Os and Ir.
14. The method according to any preceding item, wherein the metal is selected from Cu, Ni, Ag, Au, Pt, Pd, Al, Fe, Co, Ti, Zn, In, Sn and Ga.
15. The method according to any preceding item, wherein the metal is selected from Au, Ag, Pd, Cu, Mn or any alloy thereof.
16. The method according to any preceding item, wherein the metal precursor is a metal salt.
17. The method according to any preceding item, wherein the metal precursor is a metal salt of Au, Ag, or Pd.
18. The method according to any preceding item, wherein the metal salt is selected from AgNO₃, PdCl₂, PtBr₂, PtCl₂, PrCl₄, H₂Pt(OH)₆, HAuClH₄, NaAuClH₄, Pt(NH₃)₂Cl₄AuCl₂, Ag(NH₃)₂]Cl, [Ag(S₂O₃]Cl, [Ag(CN₂)]Cl, CH₃COOAg, AgF₂, AuCl, AuCl₂, AuCl₃, Au(OH)₃, KAuCl₄ and AuBr₃.
19. The method according to any preceding item, wherein the pH-adjusting agent is an acid or a base, a solution containing an acid or a base or a buffer solution of a specific pH.
20. The method according to any preceding item, wherein the pH-adjusting agent is a buffer.
21. The method according to any preceding item, wherein the buffer is selected from borate, glycine, sodium acetate, citrate buffer, phosphate-citrate and glycine-sodium hydroxide buffer.
22. The method according to any preceding item, wherein the pH-adjusting agent is a 0borate buffer, the glycoprotein is PGM or Q-mucin and the metal precursor is a silver metal precursor.
23. The method according to any preceding item, wherein the pH-adjusting agent is a glycine buffer, the glycoprotein is PGM or Q-mucin and the metal precursor is a gold metal precursor.
24. The method according to any preceding item, wherein the pH-adjusting agent is a borate buffer, the glycoprotein is PGM or Q-mucin and the metal precursor is a palladium metal precursor.
25. The method according to any preceding item, wherein the glycoprotein is M-Qmucin, the metal precursor is a silver metal precursor and the pH-adjusting agent is a borate buffer.
26. The method according to any preceding item, wherein the glycoprotein is PGM, the metal precursor is a silver metal precursor and the pH-adjusting agent is a borate buffer.
27. The method according to any preceding item, wherein the glycoprotein is M-Qmucin, the metal precursor is a gold metal precursor and the pH-adjusting agent is a glycine buffer.
28. The method according to any preceding item, wherein the glycoprotein is PGM, the metal precursor is a gold metal precursor and the pH-adjusting agent is a glycine buffer.
29. The method according to any preceding item, wherein the glycoprotein is PGM, the metal precursor is a palladium metal precursor and the pH-adjusting agent is a borate buffer.
30. The method according to any preceding item, wherein the glycoprotein is M-Qmucin or PGM, the metal precursor is a combination of two or more metal precursors and the pH-adjusting agent is a borate buffer or a glycine buffer.
31. The method according to any preceding item, wherein the metal particles are selected from nanoparticles and microparticles.
32. The method according to any preceding item, wherein the nanoparticles are of a size of between 20 and 50 nm.
33. The method according to any preceding item, wherein the microparticles are of a size of between 1 and 1.5 microns.
34. The method according to any preceding item, wherein the metal particles are a combination of nanoparticles and microparticles.
35. The method according to any preceding item, wherein the combination comprises particles of sizes ranging from 5 to 1,500 nm (1.5 micron).
36. The method according to any preceding item, wherein the metal particles are in an aggregated or non-aggregated form.
37. The method according to any preceding item, wherein the metal particles are spherical, substantially spherical, trigonal or hexagonal.
38. The method according to any preceding item, further comprising a step of separating the metal particles from the glycoprotein.
39. A method of synthesis of metal particles, the method comprising causing reduction of at least one metal precursor, under pH-dependent conditions, in a biological matrix comprising at least one glycoprotein, wherein the pH-dependent conditions affect at least one conformational change in the glycoprotein, to thereby control the particles shape, size and aggregation.
40. A method of producing metal particles, the method comprising affecting a change in a conformational state of at least one glycoprotein enriched with a metal precursor by adjusting/altering the pH of the at least one glycoprotein, thereby causing reduction of the metal precursor to a metal particle.
41. A method of selective synthesis of metal particles, the method comprising treating a complex of at least one glycoprotein and a metal precursor under selected pH conditions to thereby selectively produce metal particles of a predetermined size, shape and aggregation.
42. A glycoprotein/metal particle complex obtained according to a method of any one of items 1 to 41.
43. A glycoprotein/metal particle complex comprising:
   Q-mucin and a population of gold particles, wherein the particles are of a size ranging between 0.5 μm and 1.5 μm and/or between 10 nm andlOOnm; or
   PGM and a population of gold particles, wherein the particles are of a size ranging between 0.5 μm and 1.5 μm and/or between 10 nm and 100 nm in size.
44. A film comprising a glycoprotein/metal particle complex according to item 42 or 43.
45. A glycoprotein/metal particle complex according to item 42 or 43, or a film according to item 44, for use as an antibacterial material.
46. A glycoprotein/metal particle complex according to item 42 or 43, or a film according to item 44, for use in a method of water purification or water desalination.
47. A glycoprotein/metal particle complex according to item 42 or 43, or a film according to item 44, for use as a catalyst.
48. An antibacterial material comprising a glycoprotein/metal particle complex according to item 42 or 43.
49. A desalination agent comprising a glycoprotein/metal particle complex according to item 42 or 43.
50. A catalyst comprising a glycoprotein/metal particle complex according to item 42 or 43.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D depict: FIG. 1A—main structural components of PGM proteins. FIG. 1B—PGM protein schematic structure. FIG. 1C—PGM unfolded structure under acidic pH. FIG. 1D—PGM folded structure under alkaline pH.

FIGS. 2A-G provide a schematic representation of the proposed mechanism of gold particles formation in PGM glycoprotein. FIG. 2A—PGM structure in neutral pH, FIG. 2B—PGM structure in neutral pH after addition of gold ions, FIG. 2C—PGM-gold complex in acidic pH, FIG. 2D—PGM-gold complex in alkaline pH, FIG. 2E—Gold nanoparticles formed in acidic pH, FIG. 2F—Gold nanoparticles formed in alkaline pH, FIG. 2G—PGM structure schematic.

FIGS. 3A-B provide: FIG. 3A—PGM-Gold nanoparticles complex solutions in different pH buffers, from left to right pH3, pH6 and pH9, FIG. 3B—Marine mucine-Gold nanoparticles complex solutions in different pH buffers, from left to right pH3,pH6 and pH9.

FIG. 4 provides UV-Vis absorption spectra for samples of PGM-AuNp complex in X-6=pH3, X-7=pH6, and X-8=pH9 in water from 250 840 nm.

FIGS. 5A-B present (FIG. 5A) Gold nano triangle synthesized by PGM and its energy-dispersive X-ray spectrum (FIG. 5B).

FIGS. 6A-F depict PGM glycoprotein-AuNp complex in different pH environments. FIG. 6A and FIG. 6B—PGM-AuNp complex in pH=3, FIG. 6C and FIG. 6D—PGM-AuNp complex in pH=6, FIG. 6E and FIG. 6F—PGM-AuNp complex in pH=9.

FIGS. 7A-F depict marine mucin glycoprotein (M-mucin)-AuNp complex in different pH environments. FIG. 7A and FIG. 7B- M-mucin-AuNp complex in pH=3, FIG. 7C and FIG. 7D—M-mucin-AuNp complex in pH=6, FIG. 7E and FIG. 7F—Q-mucin-AuNp complex in pH=9.

FIGS. 8A-F depict the synthesis of gold nanoparticles on M-mucin solid films in different pH environments. FIG. 8A and FIG. 8B—M-mucin film -AuNp complex in pH=3, FIG. 8C and FIG. 8D—M-mucin film-AuNp complex in pH=6, FIG. 8E and FIG. 8F—M-mucin film-AuNp complex in pH=9.

FIGS. 9A-D depict the synthesis of gold nanoparticles on M-mucin nanofibers in pH=3. FIG. 9A and FIG. 9B—M-mucin nanofibers scaffold with dipped into M-mucin-AuNp complex pH=3, FIG. 9C and FIG. 9D—synthesis of AuNp on M-mucin nanofibers in pH=3.

FIG. 10 provides temperature measurements of solutions containing samples of PGM+AuNp XL3-XL4 (pH9) and XL5-XL8 (pH3) as a function of irradiation time using a NIR laser at 808 nm and 1.25 W/cm3. Samples were irradiated for 50, 100, and 200 seconds from room temperature. Measurements were carried out in triplicates (n=3).

FIG. 11 provides temperature measurements of irradiation by NIR laser at 808 nm and 1.25W/cm3 of solid film of X-6 sample dried on glass slide.

FIG. 12 provides water condensation under irradiation of 808 nm laser.

FIG. 13 provides weight loss vs. time of DI reference and PGM-AuNp samples (XL8, XL8 sponge, LS₃, NF LS₃).

FIG. 14 A-B provides water evaporation rate in the presence and absence of gold nanoparticles. FIG. 14A—under solar simulator (1kW/m²): water mass as a function of time. Yellow line-pH7, red line-water only, green: pH4, 30 mg, blue: pH4, 70C. FIG. 14B—under sun light: blue: only water, green: pH7, orange: pH4, 30 mg, dark green: pH4, 70° C.

FIG. 15A-C depict the synthesis of gold nanoparticles in PGM under various pH conditions. FIG. 15A—image of the solutions in the pH range of 2-10 (increase in pH from left to right). FIG. 15B—UV-vis spectra of the PGM+AuNp complexes at the different pH (2-10). FIG. 15C—temperature measurements of solutions containing samples of PGM+AuNp at pH 2-10 as a function of the pH using a NIR laser at 808 nm and 4W after 10 min of irradiation.

FIG. 16A-C depict the synthesis of gold nanoparticles in different PGM mass (10-90 mg). FIG. 16A—image of the solutions PGM mass increases from left to right. FIG. 16B—UV-vis spectra of the PGM+AuNp complexes at the different PGM mass. FIG. 16C—temperature measurements of solutions containing samples of PGM+AuNp in different PGM mass as a function of the pH using a NIR laser at 808 nm and 4W after 10min of irradiation.

FIG. 17A-C depict the synthesis of gold nanoparticles in PGM at different Au concentrations. FIG. 17A—image of the solutions [Au] increases from left to right. FIG. 17B—UV-vis spectra of the PGM+AuNp complexes at different Au concentrations. FIG. 17C—temperature measurements of solutions containing samples of PGM+AuNp at different Au concentrations as a function of the pH using a NIR laser at 808 nm and 4W after 10min of irradiation.

### DETAILED DESCRIPTION OF EMBODIMENTS

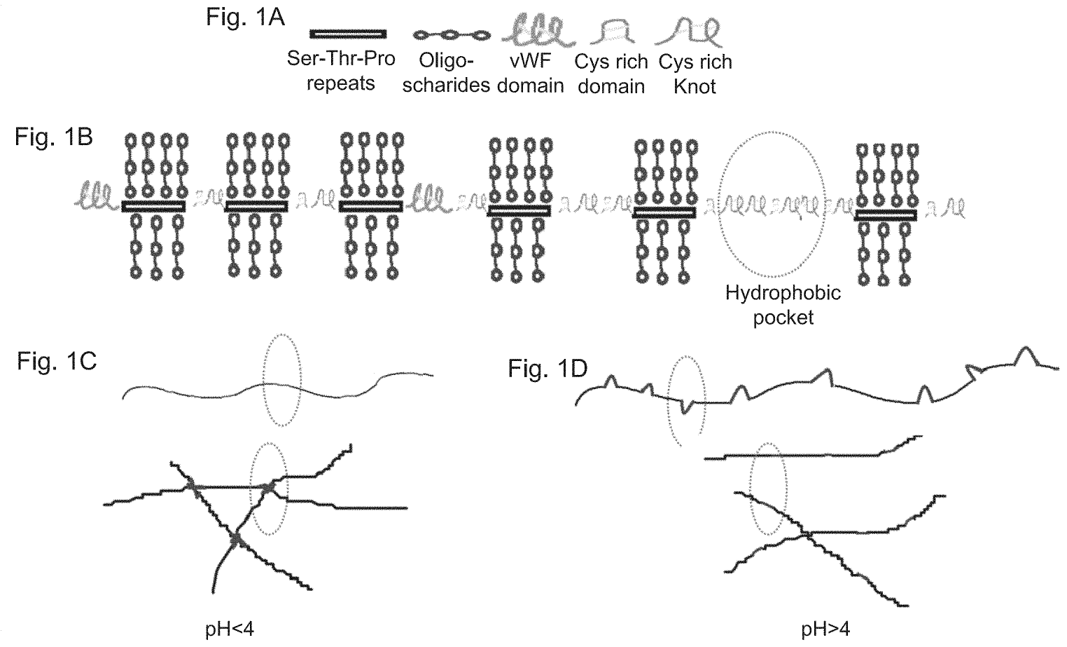

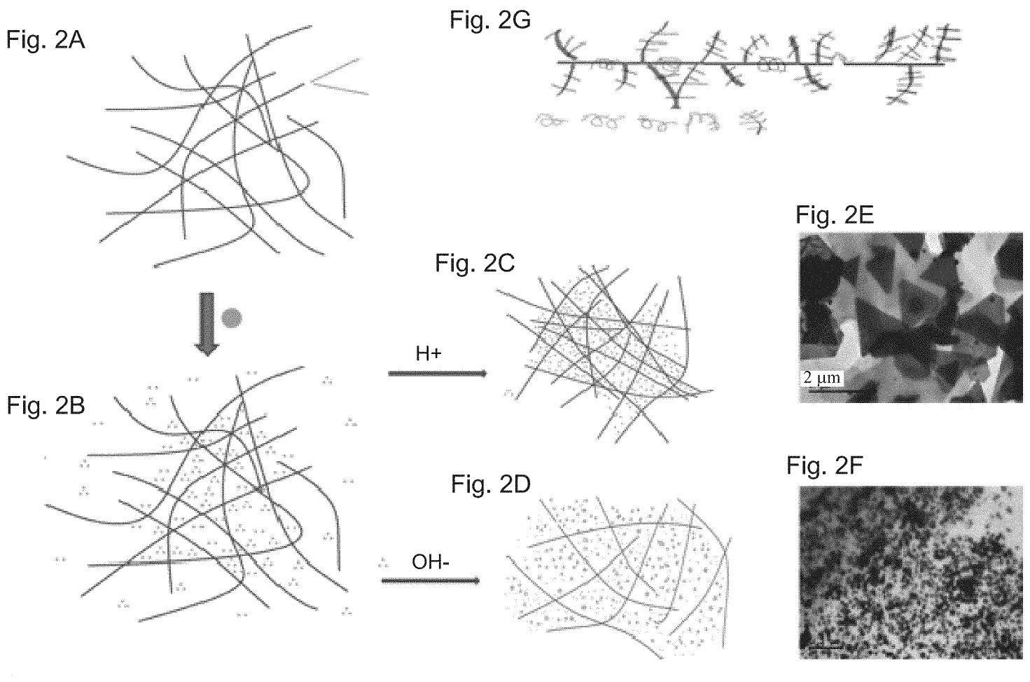

FIG. 1 provides depiction of mucin conformational forms. The pH depended formation of various gold nanoparticles structures in PGM protein matrix may be explained by the mechanism show in FIG. 2. The reducing active group of mucin glycoprotein mainly located in the hydrophobic regions along the main protein chain. Those hydrophobic domains mainly consist from cysteine amino acids with active thiols groups (FIG. 2G). During the exposure of mucin protein to protons or hydroxyls the protein changes its configuration by exposing the hydrophobic regions (acidic conditions) (FIG. 2C) or on the contrary keeping them closed with stabilized salt bridges between negative and positive charged amino acids (alkaline conditions) (FIG. 2D). The exposure of the hydrophobic regions allows hydrophobic interactions between mucin individual proteins leading to formation of dense gel networks of mucin units. On the other hands when the hydrophobic regions are closed the mucin protein sub units interact with each other only via electrostatic level leading to loose structure with considerably larger space between the individual mucin units. When the gold ions are mixed with mucin glycoprotein in neutral pH (FIG. 2B) they are entering the hydrophobic domains and begin the nucleation process forming the gold seeds which act as precursor for future nanoparticles. With addition of protons the hydrophobic domains are opening allowing hydrophobic interactions between the mucin units. This promotes contact between the gold seeds located in diverse mucin units by this allowing formation of hexagonal and trigonal particles in newly formed hydrophobic domains (FIG. 2E) and circular particles (FIG. 2F) in closed hydrophobic domains. Addition of hydroxyls promotes repulsion of the mucin units by electrostatic forces by this maintaining the hydrophobic domains enclosed limiting the interaction between them. Those limited protein interactions promotes formation of dispersed circular nanoparticles.

Apparently the pH conditions effect not only the PGM conformation but also the reduction reaction kinetics. In alkaline conditions the reduction reaction proceeds in much faster rate than in acidic pH. This may be explained by the close proximity of thiol units in closed hydrophobic domains to the gold seeds which induces the reduction process and buildup of the gold circular nanoparticles. In acidic pH the distance between the thiols is larger by this allowing slower buildup of gold particles by this allowing formation of more complex hexagon and triangular nanostructures and microstructures.

Example of Process Including Materials, Process and Results:

Materials:

In the present invention the next materials were used:

AgNO3 (sigma), HAuClH4 (sigma), NaAuClH4(sigma), PdCl2 (sigma), Porcine Gastric Mucin (PGM) (sigma), Marine Mucin, Hydrochloric acid (sigma), Sodium Hydroxide (sigma), Glycine(sigma), Ethanol anhydrous (Merck), Aqua regia, PCL(sigma)

Synthesis of Ag Nanoparticles—XJF, PGM sol

Typical synthesis of Ag nanoparticles in mucin protein matrix involves the next steps:

Appropriate Mucin protein (M-Qmucin/PGM) is weighted with typical protein weight is between 10-50 mg in lyophilized form or 0.5-1 gram in non-lyophilized form. The mucin protein is dissolved in 3 ml of AgNO₃ solution that can be between 2.5*10⁻⁵ M to 2.5*10⁻³ M. After lhr of stirring borate buffer of pH=9 is added to the Mucin-Ag+ solution. The Mucin-Ag+ solution is left for stirring in dark till completion of the reaction. Typical synthesis time is between 48-72 hr depending on silver salt concentration and on mucin type.

Synthesis of Au Nanoparticles—M-Qmucin, PGM sol

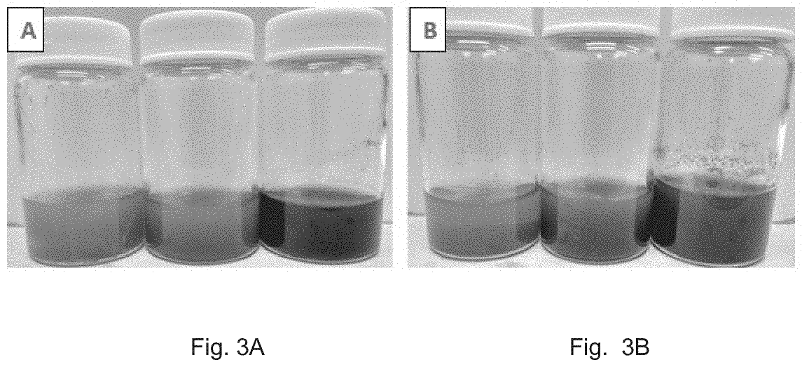

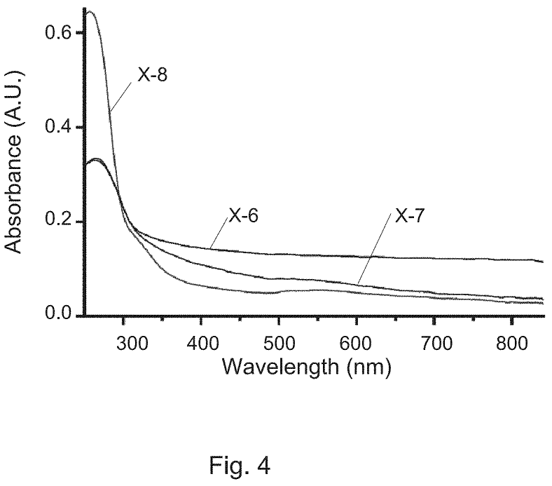

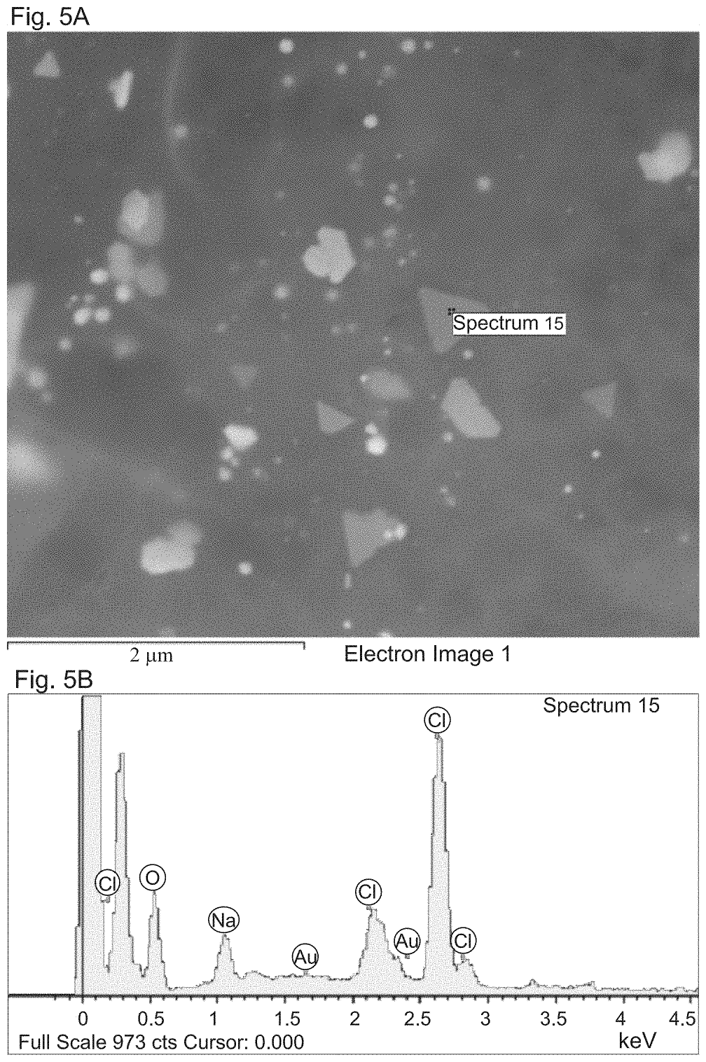

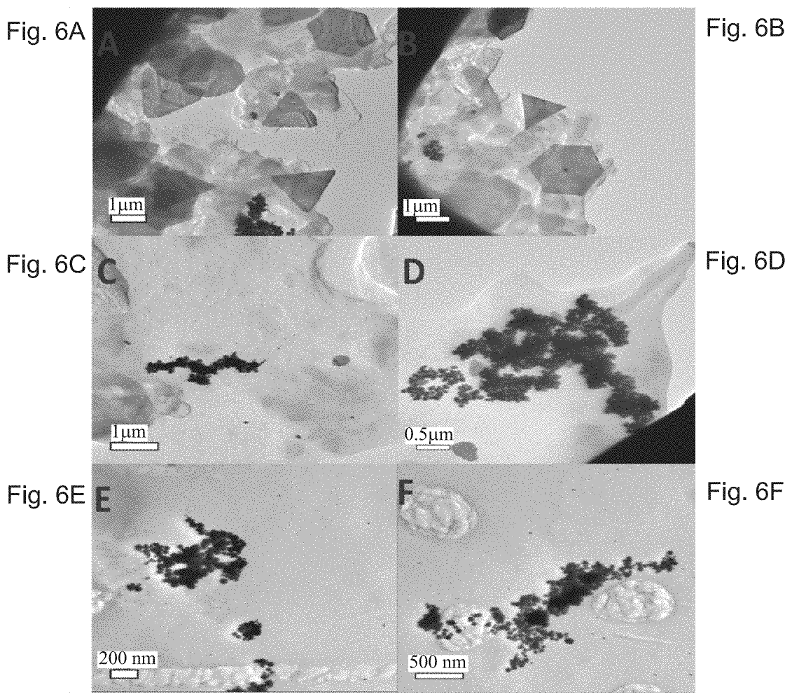

Appropriate Mucin protein (M-Qmucin/PGM) is weighted with typical protein weight is between 10-50 mg in lyophilized form or 0.5-1 gram in non-lyophilized form. The mucin protein is dissolved in AuClH₄ solution which volume can 2.5 ml to 5 ml that can be between 2.5*10⁻⁵ M to 2.5*10⁻³ M and stirred for lhr. To the previously dissolved Mucin protein-gold ions solution we add Glycine buffer in volume that can be between 2.5 ml-5 ml with appropriate pH value (3, 6, 9). After the addition of appropriate buffer the complex solution is purged from oxygen by addition of ambient nitrogen gas and sealed with parafilm. The reaction solution is stirred in dark in 45 degrees for 48 hr-72 hr till appropriate color appears (FIGS. 3A and 3B). Glycine buffer pH is responsible for Mucin conformational changes which have direct effect on synthesized gold nanoparticles size, shape, diffraction and optical properties (FIG. 4). Mucin-Au+complex in glycine buffer of pH=3 results in synthesis of mostly triangular (FIG. 5) and hexagonal particles (FIG. 6A and 6B) with size range between 0.5 μm-1.5 μm and circular dispersed nanoparticles with size range between 10 nm-100 nm. Mucin-Au+complex in glycine buffer pH=6 results in circular nanoparticles with mild aggregation and size range between 20 nm-50 nm (FIG. 6C and 6D). Mucin-Au+ complex in glycine buffer pH=9 results in circular nanoparticles in heavy aggregative state and size range 20 nm-50 nm (FIG. 6E and 6F).

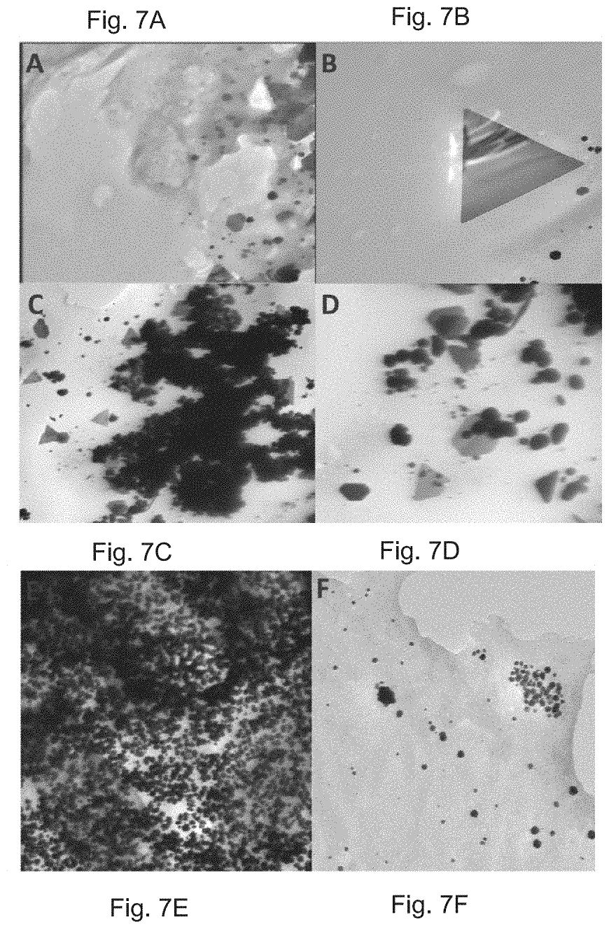

Similar HR-TEM observations were made in synthesis of Au nanoparticles in M-Qmucin proteins (FIG. 7).

Synthesis of Au Nanoparticles Mucin Solid Film

Typical synthesis of Au nanoparticles on mucin solid protein film involves two preparative steps. First the mucin solid protein film is prepared by drying the M-Qmucin protein gel/paste under fume hood or vacuum until formation of solid uniform film.

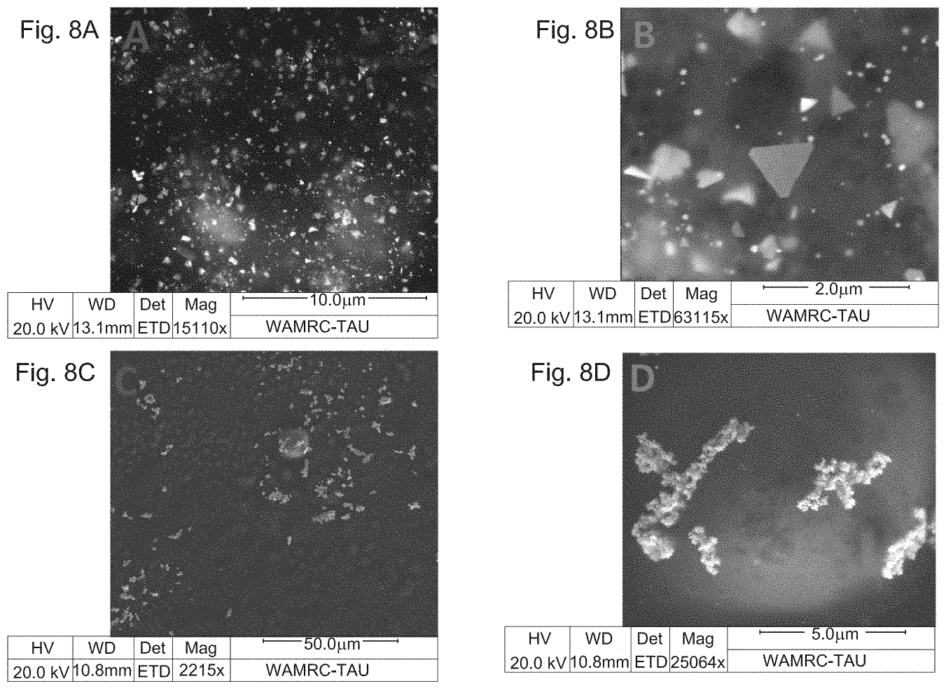

Second the mucin solid protein film with typical minimum weight of 50-100 mg is added to AuClH₄ solution of 2.5*10⁻⁵ M to 2.5*10⁻³M and stirred till gold ions are absorbed into the film (1-2 hr). After the Au ions absorbance into the film, 3 ml of glycine buffer is added in pH values 3-9 in order to synthesize Au nanoparticles with different shapes as previously described (FIG. 8). After stirring the mucin film-Au complex for 48 hr in RT the film can be dried under fume hood or solubilized in heated strong acid for example 1M HCl or Acetic acid.

Synthesis of Au/Ag Nanoparticles on Mucin Nanofibers

Typical synthesis of Au nanoparticles on Mucin nanofibers involves two primary steps:

First is the Mucin nanofibers preparation and second synthesis of Au or Ag nanoparticles on the structural matrix of Mucin nanofibers.

In order to prepare Mucin nanofibers, 50 mg of pristine Mucin protein or mixture of Mucin protein and other bio polymers such as collagen, hyaluronic acid, cellulose, gelatin are added to a carrier solvent that can be acetic acid, aqua regia, HFIP, TFA, acetic acid/ethanol, acetic acid/chloroform and others and stirred until full solubilization.

After the solubilization of the mucin proteins the stabilizing co-polymer is added in appropriate ratio to the protein that can be from 10%/90% co-polymer/protein mass to 50%/50% co-polymer/protein mass and stirred till full solubilization of the co-polymer in the carrier solvent.

The co-polymers that can be used in the process of the formation of the mucin nanofibers are: poly-caprolactone (PCL), poly vinyl alcohol (PVA), poly-lactic acid (PLA), sodium alginate, poly styrene and others.

The mucin protein-co polymer complex solution is then loaded into electrospinning setup and ran under various electrospinning conditions such as electrode distance, solution flow speed and applied voltage. The electrospinning conditions also heavily depend on the co-polymer type. Typical electrospinning conditions for an example with co-polymer PCL include: flow speed: 3 microliter/minute, electrode distance: 24 cm, voltage:14 kV. The average diameter of the formed Mucin nanofibers is 200-300 nm with porosity of 25%-35%.

The second step is synthesis of metal nanoparticle on the nanofibrous matrix of Mucin nanofibers. In typical synthesis procedure, the mucin nanofiber scaffold is cut into rectangular scaffolds of 2×2 cm and washed with DI in order to remove any residual solvent traces. Afterwards the mucin scaffolds are put into 2 ml Au ions and 3 ml of appropriate buffer OH solution and stirred for 24 hr in dark.

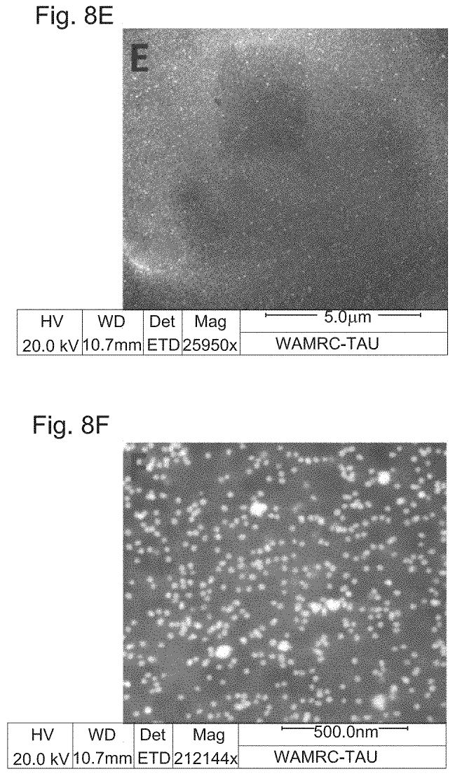

The shape and size of the nanoparticles (FIG. 9) have clear indication of pH dependency as in gold nanoparticles synthesis in solution. Mucin nanofibers and gold ions in acidic conditions lead to synthesis of triangular and hexagonal nanoparticles. synthesis in neutral and alkaline pH leads to creation of circular gold nanoparticles concentrated in aggregates and spread on nanofibers surface.

Synthesis of Pd Nanoparticles in Mucin Protein

Typical synthesis of Pd nanoparticles in mucin protein matrix involves the next steps: Appropriate Mucin protein is weighted with typical protein weight is between 10-50 mg in lyophilized form, or 0.5-1 gram in non-lyophilized form. The mucin protein is dissolved in 3 ml of PdCl₂ solution that can be between 2.5*10⁻⁵ M to 2.5*10⁻³ M. After lhr of stirring borate buffer of pH=9 is added to the Mucin-Pd+solution. The Mucin-Pd+ solution is left for stirring in dark till completion of the reaction. Typical synthesis time is between 48-72 hr depending on Palladium salt concentration and on mucin type.

Synthesis of Alloy Nanoparticles in Mucin: Pd—Au, Pd—Ag, Au—Ag

Synthesis of alloy nanoparticles of Pd—Au, Pd—Ag, Au—Ag in mucin proteins follows the same synthesis protocol of stand-alone synthesis of metal nanoparticle in mucin with several additional steps. After completion of synthesis of Au/Ag/Pd Np in mucin protein in order to synthesizes desired alloy nanoparticle we add 1 ml of 2.5*10^{-31 3} of Ag/Au/Pd metal ion solution. The complex solution is stirred in RT for 48 hr until completion of the reaction.

Optical and Hyperthermia Measurements of Au Nanoparticles Synthesized By Mucin Proteins

Samples of PGM+Au nanoparticles in different pH conditions X-6=pH3, X-7=pH6, and X-8=pH9 were prepared at a concentration of 1.7 mM Au in deionized water. In order to determine their optical properties, sample solutions were diluted by a factor of 10, and placed in plastic cuvettes with a beam path length of 10 mm UV-Vis spectra were recorded accordingly in a Nanodrop™2000c fitted with a cuvette reader (Thermo Scientific, Australia). The spectra for each sample were measured from 250 to 840 nm (FIG. 4).

From FIG. 4 it may be concluded that each of the samples exhibited strong absorption at 280 nm, usually associated with aromatic rings in tyrosine and tryptophan amino acids comprising the surface protein layer. Additionally, broad peaks centered around 580 nm for X-7 and X-8 were associated with plasmon resonance effects originating from the 20 nm Au nanoparticles, while no such peak was observed for the sample X-6. Finally, absorption at 808 nm was much more intense for X-6 as opposed to samples X-7 and X-8.

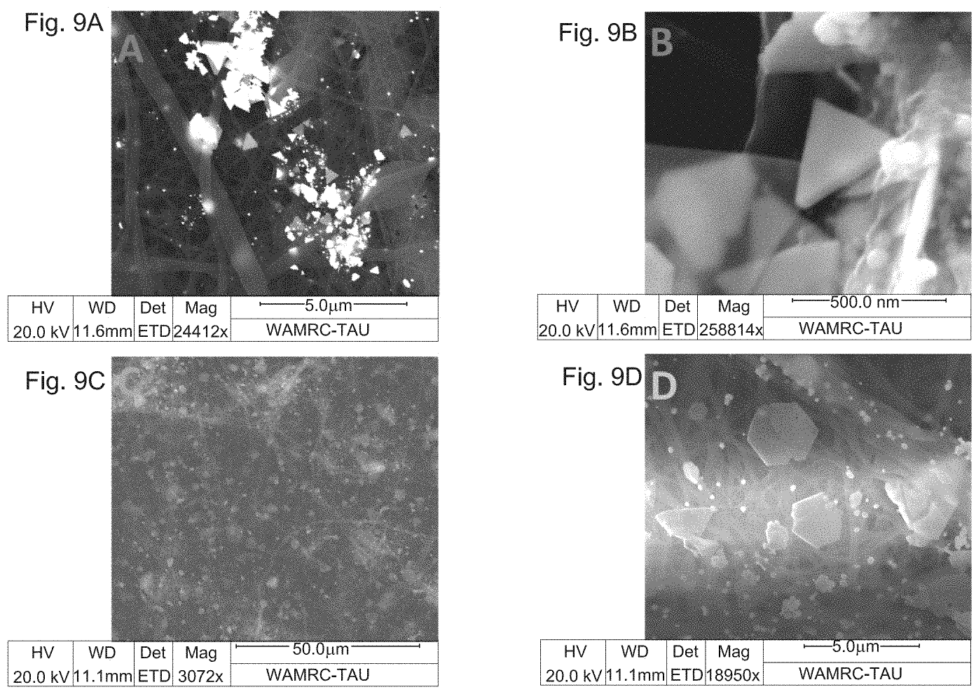

The samples were then irradiated with an 808 nm continuous wave diode laser at a power density of 1.25 W/cm³ in water for 50, 100, and 200 sec (FIG. 10). The temperature of the solutions was measured pre- and post-irradiation using a FLIR (i7) thermo-imaging camera (n=3). Results showed a drastic increase in temperature for XL-5,XL-6,XL-7 and XL-8 after irradiation for 50 sec from room temperature to 90-100° C. and maximum of 120° C. after 200 sec. Heating effect was less drastic for XL-3 and XL-4 which temperature increase was measured at 40° C. for XL-3 and 60° C. for XL-4 after 50 sec and reached a maximum temperature of 58° C. and 90° C. for XL-3 and XL-4 respectfully. The higher heating effect value was attributed to the enhanced absorption of XL-5 . . . XL-8 samples in the NIR region of the spectrum (800 1200) as is shown in FIG. 10. Given that the [Au]=1.7 mM for all samples, the difference in NIR absorption was attributed purely to particle geometry, shape, and size.

Samples of PGM+Au nanoparticles in different pH conditions, Au concentration, and PGM mass were used for optimization of the hyperthermia effect under laser 808 nm irradiation. The solution that provide the most drastic heating effect will be considered as the optimized synthesis procedure. In order to determine their optical properties, samples were diluted and placed in plastic cuvettes with a beam path length of 10 mm UV-Vis spectrums were recorded as previously described. The spectra for each sample was measured from 300 to 1000 nm. The samples were irradiated with an 808 nm continuous wave diode laser at a power density of 4 W in water for 10min The temperature of the solutions was measured pre- and post-irradiation using a thermocouple.

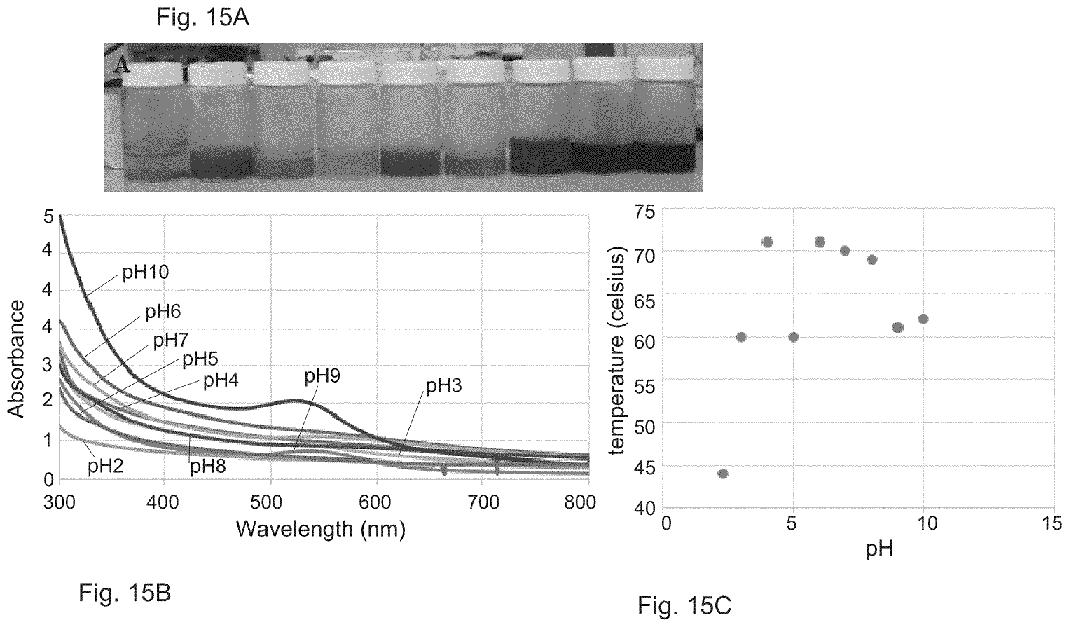

Solutions with pH 2-10 were prepared at a concentration of [Au]=1.25 mM in deionized water (FIG. 15A). The samples under pH conditions of pH=9, 10 exhibited strong absorption centered around 550 nm, whereas pH=2, 3, 7, 8 exhibited a broad weak peak centered around 550 nm associated with plasmon resonance effects. The differences in the intensity of the peaks is associated with the particles average size, which is smaller for the particles with the broad and weaker peaks. No such peak was observed for the samples pH=4, 5, 6 (FIG. 15B). Finally, heating effect at 808 nm was the most intense for pH=4, 6 (FIG. 15C).

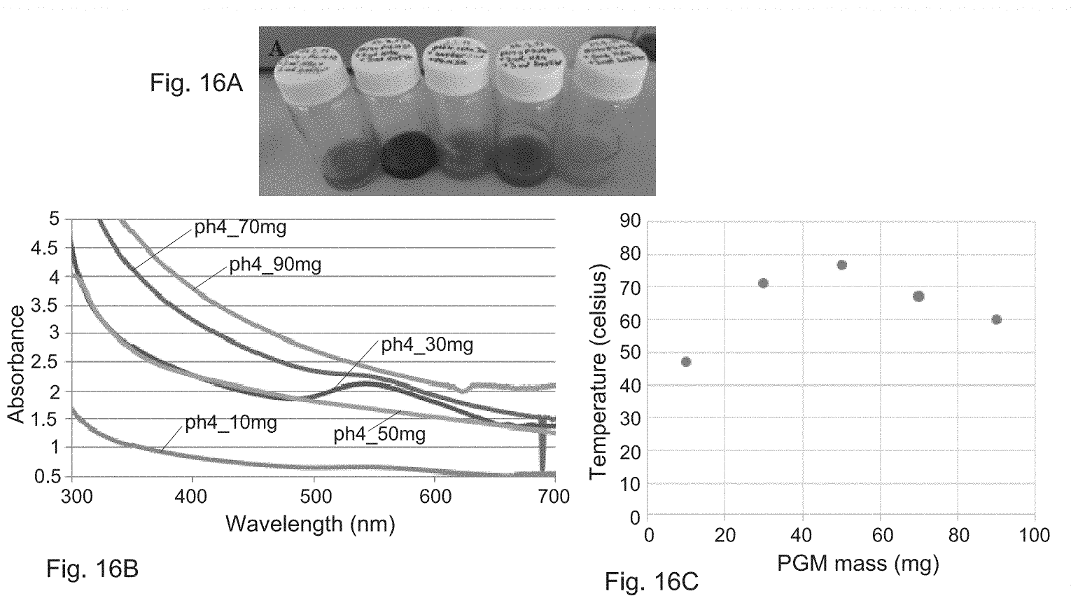

Later, a range of PGM mass (10-90 mg) with an identical amount of water at pH=4 and [Au]=1.25 mM were prepared (FIG. 16A). The sample of pH4 with 30 mg of PGM exhibited strong absorption centered around 550 nm, 70 mg and 10 mg of PGM exhibited a broad weak peak centered around 550 nm associated with plasmon resonance effects, whereas no peak was excepted for 50 mg and 90 mg of PGM (FIG. 16B). Finally, heating effect at 808 nm was the most intense for pH4, 50 mg of PGM (FIG. 16C).

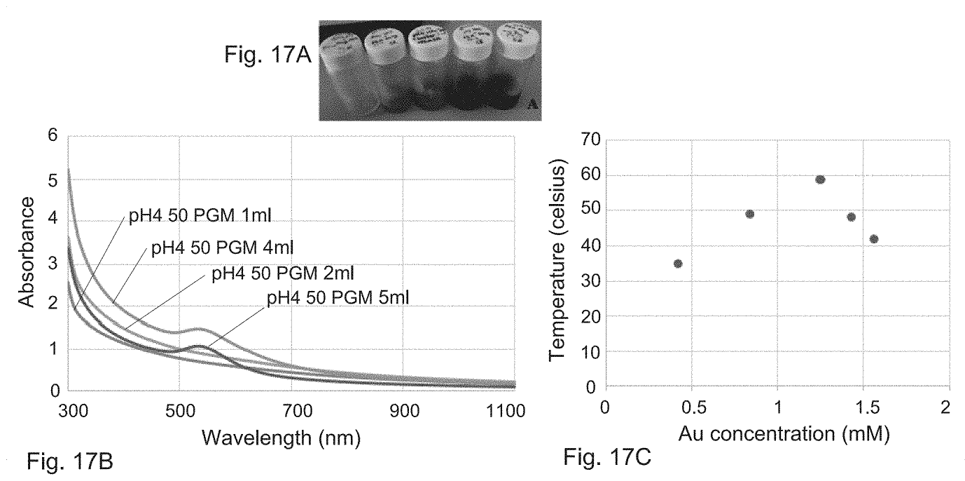

Finally, a range of Au concentrations, 0.42-1.56 mM, with 50 mg PGM at pH=4 and [Au]=1.25 mM were prepared (FIG. 17A). The samples of pH4 with 4-5m1 of Au (1.43 and 1.56 respectively) exhibited strong absorption centered around 550 nm, whereas no peak was excepted for the three lower concentrations (FIG. 17B). Finally, heating effect at 808 nm was the most intense for [Au]=1.25 mM (FIG. 17C).

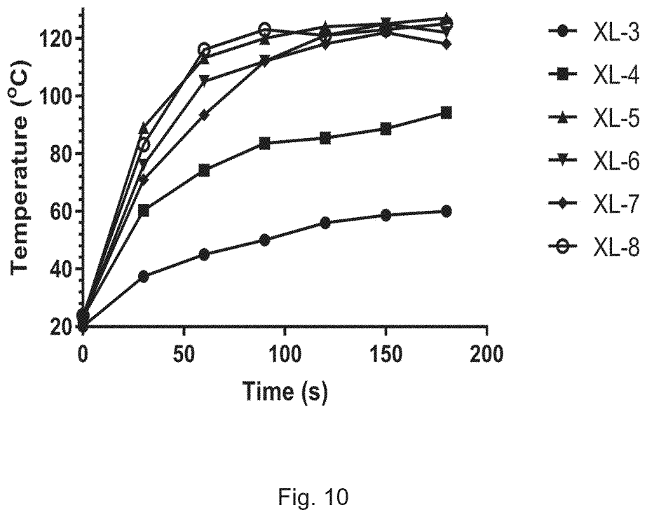

Additional hyperthermia measurement experiment in solid state was performed on sample XL-3 which was dried on an glass slide. The experiment showed that in solid state the X-6 can go through several cycles of heating without any damage to the protein sample (FIG. 11).

Water Condensation Experiments Under Laser Irradiation (808 nm)

Samples with PGM-gold Np complex were put in DI in 5%/95% (PGM/DI) ratio and irradiated for duration of 15 minutes by 808 nm NIR laser.

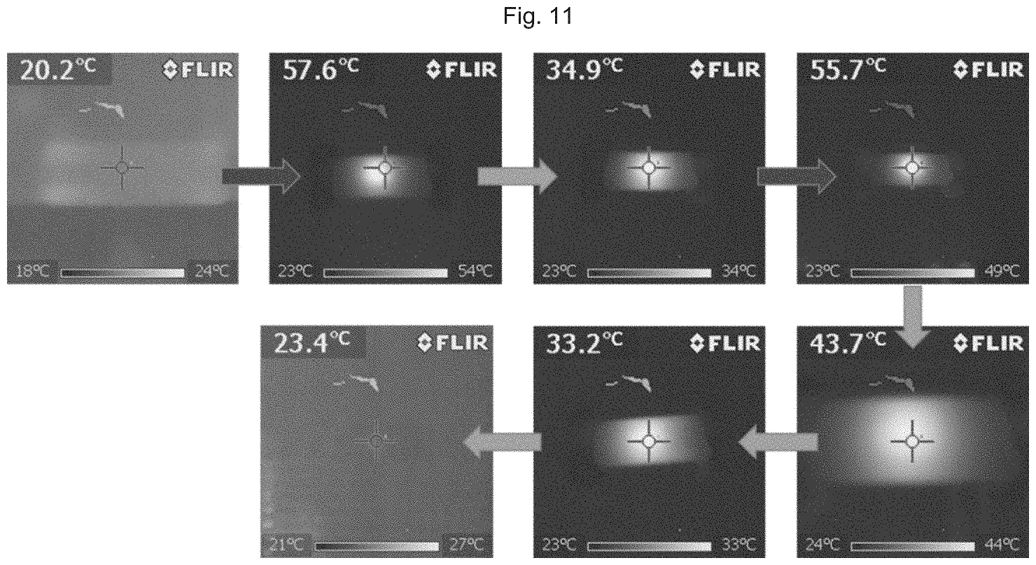

Before, during and after the laser treatments both PGM-AuNp and DI reference were weighted and the mass loss to water condensation was calculated (FIG. 12). From the experimental results we calculated that the water condensation of PGM-AuNp samples was higher by 30% then in referenced DI sample.

Water Condensation Under Solar Lamp

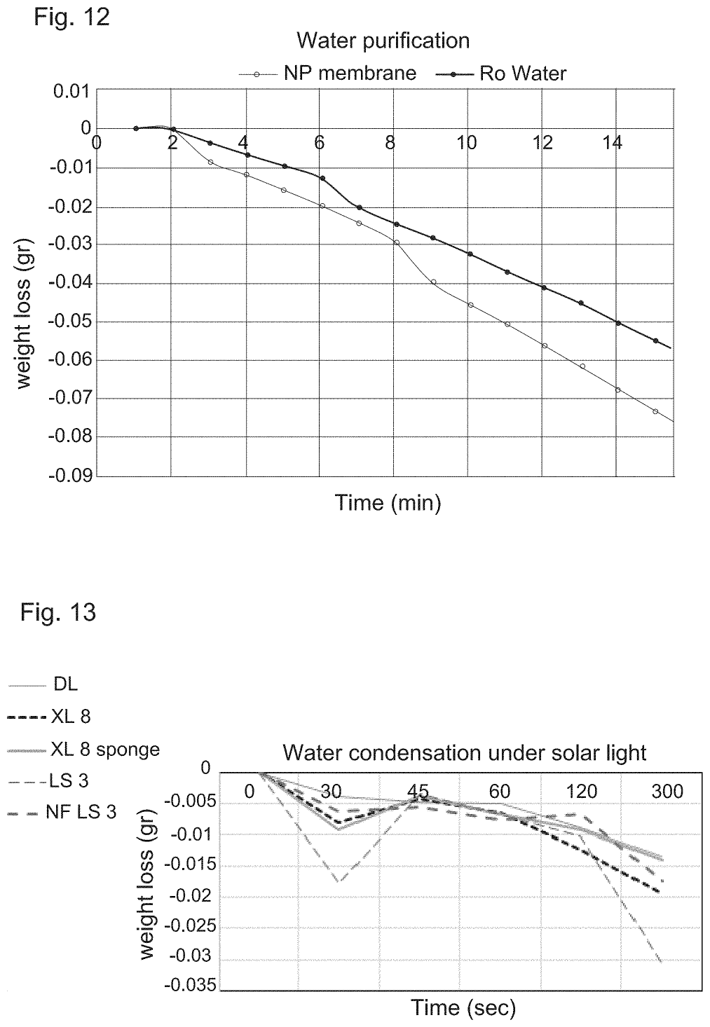

Several samples of PGM-AuNp complexes diluted in DI water or inserted into carrier membrane were put under solar simulator (450W, 1.5 AM) and irradiated with solar light for different time durations (30sec, 45 sec, 60 sec, 120 sec and 300 sec). PGM-AuNp and DI reference were weighted before and after solar irradiation and the mass loss to water condensation was calculated (FIG. 13). From the experimental results we calculated that the water condensation of PGM-AuNp samples(XL8,XL8 sponge, LS₃ and NF LS₃) was higher by 20%-92% then in referenced DI sample depending on PGM-AuNp type and concentration (Table 1).

TABLE 1
Weight loss under solar irradiation

| | DI | | XL8 | | |
|---|---|---|---|---|---|
| Time (sec) | water | XL8 | sponge | LS3 | NF LS3 |
| 0 | 0 | 0 | 0 | 0 | 0 |
| 30 | −0.0039 | −0.008 | −0.0092 | −0.0178 | −0.0063 |
| 45 | −0.0047 | −0.0043 | −0.0038 | −0.0035 | −0.0056 |
| 60 | −0.0049 | −0.0064 | −0.0067 | −0.0068 | −0.0075 |
| 120 | −0.0089 | −0.0124 | −0.0091 | −0.0103 | −0.0067 |
| 300 | −0.0133 | −0.0195 | −0.0139 | −0.0305 | −0.0174 |
| Weight loss (gr) | 0.0357 | 0.0506 | 0.0427 | 0.0689 | 0.0435 |
| after total 9 minutes | | | | | |

TABLE 2
exemplary systems prepared according to methods of the invention

| Mucin | | | | Resulting | |
|---|---|---|---|---|---|
| glycoprotein | Metal | Buffer | pH | particle size | Resulting shape |
| M-Qmucin | Ag | Borate | 9 | | |
| PGM | Ag | Borate | 9 | | |
| M-Qmucin | Au | Glycine | 3 | 0.5 μm-1.5 μm | triangular and hexagonal |
| | | | | | particles |
| | | | | 10 nm-100 nm | circular dispersed nanoparticles |
| M-Qmucin | Au | Glycine | 6 | 20 nm-50 nm | circular nanoparticles with |
| | | | | | mild aggregation |
| M-Qmucin | Au | Glycine | 9 | 20 nm-50 nm | circular nanoparticles in heavy |
| | | | | | aggregative state |
| PGM | Au | Glycine | 3 | 0.5 μm-1.5 μm | triangular and hexagonal |
| | | | | 10 nm-100 nm | particles (Additional |
| | | | | | comments: (1) Enhance NIR |
| | | | | | absorption comparable to NP at |
| | | | | | PH = 6 and PH = 9. (2) |
| | | | | | undergo several cycles of |
| | | | | | heating without damaging the |
| | | | | | protein sample. (3) No |
| | | | | | cytotoxicity in cancer cells. (4) |
| | | | | | Enhanced water condensation |
| | | | | | under laser irradiation. (5) |
| | | | | | Enhanced water condensation |
| | | | | | under solar lamp). (6) circular |
| | | | | | dispersed nanoparticles Able to |
| PGM | Au | Glycine | 6 | 20 nm-50 nm | circular nanoparticles with |
| | | | | | mild aggregation |
| PGM | Au | Glycine | 9 | 20 nm-50 nm | circular nanoparticles in heavy |
| | | | | | aggregative state |
| PGM | Pd | borate | 9 | 5-20 nm | Circular |
| PGM | *Pd—Au | *Borate and glycine | 9 to 3 | *5-100 nm | *Circular |
| | *Pd—Ag | *Borate | 9 and 9 | *5-20 nm | *Circular |
| | *Au—Ag | *glycine and borate | 3 to 9 | *0.5 μm-1.5 μm | *Triangular and circular |
| | | | | and 5-20 nm | (Additional comments: Au—Ag |
| | | | | | antibacterial material with anti- |
| | | | | | biofilm capabilities) |

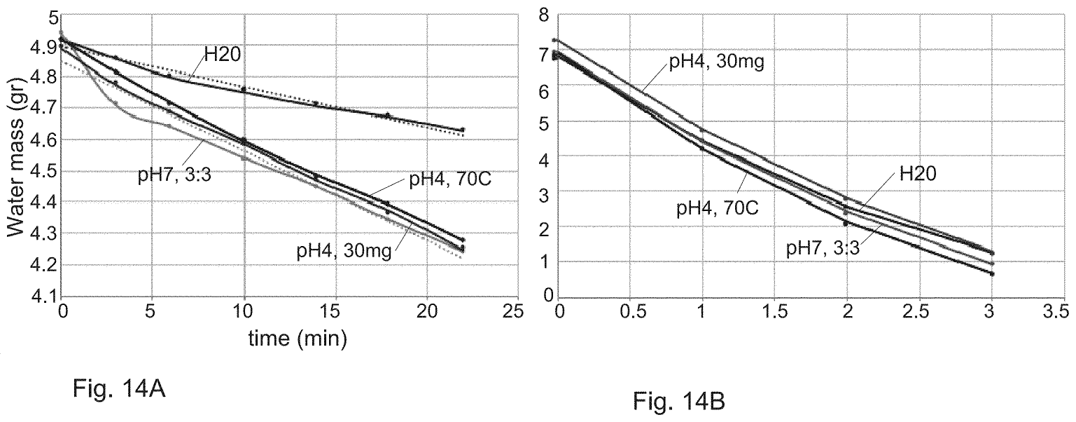

Water Evaporation Rate in Solar Simulator and Field Test Several samples of PGM-AuNp complexes were examined under solar simulator (1kW/ m²) and in field test under the sun. The solutions were diluted in DI water to identical concentration and weigh on a scale in fixed time periods for measuring water loss rate of each solution. The experiment was performed 5 times for statistics. The same samples were also examined in field tests. The experiment was performed 5 times for statistics (FIG. 14). The experimental results revealed a significant increase in the efficiency of photothermal energy conversion-reaching up to 0.80 Kg/m²h water loss under 1 sun (1kW/ m²) for particles solution in water, compared to about 0.34 Kg/m²h water loss for a system consisted only of water (Table 3).

TABLE 3
Water weight loss under solar irradiation

| Water loss | water loss rate | | |
|---|---|---|---|
| [Kg/hr*m²] | solar simulator | field test | |
| 0.796 | y = −0.0211x + 6.676 | y = −1.9961x + 6.6559 | pH 7, 3:3 |
| 0.74 | y = −0.0257x + 6.7913 | y = −1.9869x + 7.0016 | pH 4, 30 mg |
| 0.736 | y = −0.0274x + 6.7399 | y = −2.0734x + 6.5736 | pH 4, 70° C. |
| 0.337 | y = −0.0188x + 6.7378 | y = −1.8437x + 6.5298 | H₂O |

1. A method of producing metal particles of a preselected form, the method comprising reacting a combination of a glycoprotein or a complex of glycoprotein matrix and at least one a metal precursor with at least one a pH-adjusting agent, under conditions of at least pH permitting reductive transformation of said metal prec ursor to metal particles, wherein the pH-adjusting agent provides a pH between 3 and 9 and determines the fonn of the metal particle.

2. The method according to claim 1, wherein the metal particle provides a pH between 3 and 9 and is selected from particle size, shape and aggregation.

3. (caanceled)

4. The method according to claim 1, wherein the conditions permitting reductive transformation comprise selecting a reaction a temperature.

5. The method according to claim 1, wherein the pH is between 3 and 7, or between 3 and 6, or between 7 and 9.

6-7. (canceled)

8. The method of claim 4. wherein the conditions comprise reductive transformation at room temperature or at a temperature between 45 and 70° C.

9. The method according to claim 1, wherein the glycoprotein is at least one mucin.

10-11. (canceled)

12. The method according to claim 1. wherein the metal precursor is a metal salt or a metal complex.

13. The method according to claim 1, wherein the metal precursor comprises a metal atom selected from the group consisting of Ag, Au, Cu, Pd, Pt, Ni, Co, Cd, Fe, Sc, Sn, Al, Ti, V, Mn, Zn, Y, Zr, Nb, Tc, Ru, Rh, Mn, Hf, Ta, Re, In, Ga, Os, Ir, and any alloy thereof.

14-15. (canceled)

16. The method according to claim 12, wherein the metal precursor is a metal salt.

17-18. (canceled)

19. The method according to claim 1. wherein the pH-adjusting agent is selected from a group consisting of an acid or a base, a solution containing an acid or a base or a buffer solution of a specific pH.

20-21. (canceled)

22. The method according to claim 1, wherein the pH-adjusting agent is a borate buffer, the glycoprotein is porcine gastric mucin PGM), bovine submaxillary mucin (BSM or Q-mucin and the metal precursor is selected from the group consisting of silver, gold, and palladium metal precursor.

23. (canceled)

24. The method according to claim 1, wherein the metal particles are selected from the group consisting of nanoparticles, microparticles and a combination thereof.

25. The method according to claim 24, wherein the nanoparticles are of a size of between 20 and 50 mn.

26. The method according to claim 24. wherein the microparticles are of a size of between 1 and 5 microns.

27-30. (canceled)

31. The method according to claim 1. further comprising a step of separating the metal particles from the glycoprotein.

32. A method of synthesis of metal particles, the method comprising causing reduction of at least one metal precursor, under pH-dependent conditions, in a biological matrix comprising at least one glycoprotein, wherein the pH-dependent conditions affect at least one conformational change in the glycoprotein, to thereby control the particles shape, size and aggregation.

33. A method of producing metal particles, the method comprising affecting a change in a conformational state of at least one glycoprotein enriched with a metal precursor by adjusting/altering the pH of the at least one glycoprotein, thereby causing reduction of the metal precursor to a metal particle.

34. (canceled)

35. A glycoprotein/metal particle complex obtained according to a method of claim 1.

36. A glycoprotein/metal particle complex comprising:
Q-mucin and a population of gold particles, wherein the particles are of a size ranging between 0.5 μm and 1.5 μm and/or between 10 nm and 100 nm; or
PGM and a population of gold particles, wherein the particles are of a size ranging between 0.5 μm and 1.5 μm and/or between 10 nm and 100 nm in size.

37. A film comprising a glycoprotein/metal particle complex accor ding to claim 35 or 36.

38. A method for treating water comprising contacting water to be treated with a glycoprotein/metal particle complex according to claim 35.

39. A method of antibacterial treatment comprising contacting a surface to be treated with a glycoprotein/metal particle complex according to claim 35.

40. (canceled)

41. A method for treating water comprising contacting with water to be treated and a catalyst comprising a glycoprotein/metal particle complex according to claim 35.
